(19) United States
(12) Patent Application Publication
(10) Pub. No.: US 2016/0175363 A1
(43) Pub. Date: Jun. 23, 2016

(54) METHODS FOR GENERATING STEM CELL-DERIVED BETA CELLS AND USES THEREOF

(71) Applicant: President and Fellows of Harvard College, Cambridge, MA (US)

(72) Inventors: Douglas A. Melton, Lexington, MA (US); Jeffrey R. Millman, Somerville, MA (US)

(21) Appl. No.: 14/975,383

(22) Filed: Dec. 18, 2015

Related U.S. Application Data
(60) Provisional application No. 62/094,007, filed on Dec. 18, 2014.

(51) Int. Cl. A61K 35/39; C12N 5/071; A61K 9/50

(52) U.S. Cl. CPC A61K 35/39; A61K 9/50; C12N 5/0676; C12N 5/0677; C12N 2506/07; C12N 2501/40

(57) ABSTRACT

Disclosed herein are methods for generating SC-β cells, and isolated populations of SC-β cells for use in various applications, such as cell therapy.

### RELATED APPLICATIONS

This application claims the benefit of U.S. Provisional Application Ser. No. 62/094,007, filed Dec. 18, 2014, the entire teachings of which are incorporated herein by reference.

### BACKGROUND OF THE INVENTION

Diabetes affects more than 300 million people worldwide according to the International Diabetes Federation. Type 1 diabetes and type 2 diabetes involve β cell destruction and/or β cell dysfunction. Diabetic patients, particularly those suffering from type 1 diabetes, could potentially be cured through transplantation of β cells. While cadaveric human islet transplantation can render patients insulin independent for 5 years or longer, such approach is limited due to the scarcity and quality of donor islets (Bellin et al., 2012). Generating an unlimited supply of human β cells from stem cells could provide therapy to millions of patients as only a single cell type, the β cell, likely needs to be produced, and the mode of delivery is well understood: transplantation to a vascularized location within the body with immunoprotection. In addition, screening to identify novel drugs that improve β cell function, survival, or proliferation is also delayed due to limited islet supply and variability resulting from different causes of death, donor genetics, and other aspects in their isolation. As such, a steady, uniform supply of stem-cell-derived β cells would offer a useful drug discovery platform for diabetes. Moreover, genetically diverse stem-cell-derived β cells could be used for disease modeling in vitro or in vivo.

### SUMMARY OF THE INVENTION

There is a need for methods of generating stem cell-derived β (SC-β) cells. The present invention is directed toward solutions to address this need, in addition to having other desirable characteristics.

In accordance with an embodiment of the present invention, a method for generating stem cell-derived β (SC-β) cells is provided. The method includes contacting a cell population comprising endocrine progenitor cells or precursors thereof under conditions suitable for directing said cells to differentiate into SC-β cells with an effective amount of a first agent that specifically inhibits the level and/or activity of at least one activin receptor-like kinase (ALK), thereby generating SC-β cells. The resultant SC-β cells may have one or more improved properties in comparison with SC-β cells produced by the same protocol in the absence of the ALK inhibitor.

In accordance with aspects of the invention, the endocrine progenitor cells comprise PDX1-F/NKX6.1+/NEUROD1+/insulin+/glucagon−/somatostatin− cells. In accordance with aspects of the present invention, the precursors are selected from the group consisting of pluripotent stem cells, SOX17+ definitive endoderm cells, PDX1+ primitive gut tube cells, PDX1+/NKX6.1+ pancreatic progenitor cells, PDX1+/NKX6.1+/NEUROD1+ endocrine progenitor cells. In accordance with aspects of the invention, the endocrine progenitor cells are directed to differentiate into SC-β cells by contacting the endocrine progenitor cells under conditions that promote cell clustering with i) a transforming growth factor β (TGF-β) signaling pathway inhibitor and ii) a thyroid hormone signaling pathway activator to induce the in vitro maturation of at least some of the endocrine progenitor cells into SC-β cells.

In accordance with aspects of the invention, the effective amount of the first agent comprises a concentration range of between 0.1 μM and 110 μM. In accordance with aspects of the invention, the method further includes contacting the cell population with an effective amount of at least a second agent that specifically inhibits the level and/or activity of at least one ALK. In accordance with aspects of the invention, the effective amount of the second agent comprises a concentration range of between 0.1 μM and 110 μM. In accordance with aspects of the invention, the at least one ALK is selected from the group consisting of ALK1, ALK2, ALK3, ALK4, ALK5, ALK6 and ALK7. In accordance with aspects of the invention, the first agent and/or the second agent is selected from the group consisting of SB431542, DMH-1, and Alk5 inhibitor II.

In accordance with aspects of the invention, the at least one ALK is selected from the group consisting of ALK1, ALK2, ALK3, ALK4, ALK6 and ALK7. In accordance with aspects of the invention, the at least one ALK is selected from the group consisting of SB431542 and DMH-1. In accordance with aspects of the invention, the first agent and/or the second agent is not ALK5 inhibitor II. In accordance with aspects of the invention, the first agent and/or the second agent is not ALK5 inhibitor II administered at a concentration of 10 μM.

In accordance with aspects of the invention, the first agent and/or the second agent exhibits an IC₅₀ for the at least one ALK that is less than or equal to 500 nm or wherein the cells are contacted with a concentration of the first agent and/or the second agent that is equal to or greater than its IC50 value for at least one ALK. In accordance with aspects of the invention, the first agent and/or the second agent is more selective for the at least one ALK than for at least one mitogen activated protein kinase (MAPK) or other kinases.

In accordance with aspects of the invention, between at least 5% and 65% of the endocrine cells in the population differentiate into SC-β cells.

In accordance with an embodiment of the present invention, an isolated SC-β cell or population thereof generated according to the methods for generating SC-β cells described herein is provided. The isolated SC-β cell or population one of SC-β cells exhibits a glucose stimulated insulin secretion (GSIS) response both in vitro and in vivo. In accordance with aspects of the invention, an isolated SC-β cell or population thereof exhibits a stimulation index that is at least between 1.5-fold and 10-fold greater than the stimulation index of a control SC-β cell.

In accordance with aspects of the invention, an isolated SC-β cell or population thereof produces between approximately 300 uIU and 4000 uIU per 30 minute incubation at a high glucose concentration.

In accordance with aspects of the invention, an isolated SC-β cell or population thereof two weeks after transplantation into a subject in vivo releases between 3 uIU/mL and 81 uIU/mL of insulin within 30 minutes of administering 2 g/kg glucose to the subject.

In accordance with an embodiment of the present invention, a microcapsule comprising the isolated SC-β cell or population thereof encapsulated therein is provided.

In accordance with an embodiment of the present invention, a macroencapsulation device comprising the isolated SC-β cell or population thereof encapsulated therein is provided.

In accordance with an embodiment of the present invention, a cell line comprising an isolated SC-β cell is provided. The cell line stably expresses insulin.

In accordance with an embodiment of the present invention, an assay comprising an isolated SC-β cell or population thereof is provided. In accordance with an embodiment of the present invention, an assay comprising an SC-β cell line that stably expresses insulin is provided. The assays can be used for i) identifying one or more candidate agents which promote or inhibit a β cell fate selected from the group consisting of β cell proliferation, β cell replication, β cell death, β cell function, β cell susceptibility to immune attack, and β cell susceptibility to dedifferentiation or differentiation, and/or ii) identifying one or more candidate agents which promote the differentiation of at least one insulin-positive endocrine cell or a precursor thereof into at least one SC-β cell.

In accordance with an embodiment of the present invention, a method for the treatment of a subject in need thereof (e.g., in need of β cells) is provided. The method includes administering to a subject in need thereof an isolated population of SC-β cells and/or a microcapsule encapsulating an isolated population of SC-β cells. In accordance with an embodiment of the present invention, an isolated population of SC-β cells or a microcapsule comprising an isolated population of SC-β cells is used for administering to a subject in need thereof. In accordance with aspects of the present invention, the subject has, or has an increased risk of developing diabetes or has, or has an increased risk of developing a metabolic disorder.

In accordance with an embodiment of the present invention, an artificial islet or pancreas comprising SC-β cells produced according to a method described herein. The practice of the present invention will typically employ, unless otherwise indicated, conventional techniques of cell biology, cell culture, molecular biology, transgenic biology, microbiology, recombinant nucleic acid (e.g., DNA) technology, immunology, and RNA interference (RNAi) which are within the skill of the art. Non-limiting descriptions of certain of these techniques are found in the following publications: Ausubel, F., et al., (eds.), Current Protocols in Molecular Biology, Current Protocols in Immunology, Current Protocols in Protein Science, and Current Protocols in Cell Biology, all John Wiley & Sons, N.Y., edition as of December 2008; Sambrook, Russell, and Sambrook, Molecular Cloning: A Laboratory Manual, 3rd ed., Cold Spring Harbor Laboratory Press, Cold Spring Harbor, 2001; Harlow, E. and Lane, D., Antibodies—A Laboratory Manual, Cold Spring Harbor Laboratory Press, Cold Spring Harbor, 1988; Freshney, R. I., “Culture of Animal Cells, A Manual of Basic Technique”, 5th ed., John Wiley & Sons, Hoboken, N. J., 2005. Non-limiting information regarding therapeutic agents and human diseases is found in Goodman and Gilman's The Pharmacological Basis of Therapeutics, 11th Ed., McGraw Hill, 2005, Katzung, B. (ed.) Basic and Clinical Pharmacology, McGraw-Hill/Appleton & Lange; 10th ed. (2006) or 11th edition (July 2009). Non-limiting information regarding genes and genetic disorders is found in McKusick, V. A.: Mendelian Inheritance in Man. A Catalog of Human Genes and Genetic Disorders. Baltimore: Johns Hopkins University Press, 1998 (12th edition) or the more recent online database: Online Mendelian Inheritance in Man, OMIM™. McKusick-Nathans Institute of Genetic Medicine, Johns Hopkins University (Baltimore, Md.) and National Center for Biotechnology Information, National Library of Medicine (Bethesda, Md.), as of May 1, 2010, World Wide Web URL:.ncbi.nlm.nih.gov/omim/ and in Online Mendelian Inheritance in Animals (OMIA), a database of genes, inherited disorders and traits in animal species (other than human and mouse), at omia.angis.org.au/contact.shtml. All patents, patent applications, and other publications (e.g., scientific articles, books, websites, and databases) mentioned herein are incorporated by reference in their entirety. In case of a conflict between the specification and any of the incorporated references, the specification (including any amendments thereof, which may be based on an incorporated reference), shall control. Standard art-accepted meanings of terms are used herein unless indicated otherwise. Standard abbreviations for various terms are used herein.

### BRIEF DESCRIPTION OF THE DRAWINGS

These and other characteristics of the present invention will be more fully understood by reference to the following detailed description in conjunction with the attached drawings. The patent or application file contains at least one drawing executed in color. Copies of this patent or patent application publication with color drawings will be provided by the Office upon request and payment of the necessary fee.

FIG. 1A is a graph demonstrating that SC-β cells generated by contacting endocrine progenitor cells directed to differentiate into SC-β cells with an exemplary agent that specifically inhibits the level and/or activity of at least one activin receptor-like kinase (ALK), e.g., SB431542, DMH-1, Alk5i, SD208, etc.) exhibit a greater stimulation index relative to SC-β cells generated by contacting endocrine progenitor cells directed to differentiate into SC-β cells in the absence of treatment with the agent that specifically inhibits the level and/or activity of at least one ALK. Notably, whereas agents that specifically inhibited at least one ALK improved the stimulation index of the resulting SC-β cells, agents that inhibited MAPK worsened the stimulation index. In particular, SB431542 (1 μM), DMH-1 (1 μM), Alk5i (110 μM), and SD208 (1 μM) all specifically inhibit at least one ALK and are not known to detectably inhibit MAPK, whereas A8301 is both an ALK inhibitor and a MAPK inhibitor, and groups with other MAPK inhibitors, such as SB203580, SB202190 and U0126, which resulted in SC-β cells exhibiting worse stimulation indices than control SC-β cells. Stimulation index=[insulin@20 mM glucose]/[insulin@2 mM glucose]. Relative Stim Index=Stim Index/Stim Index of Control.

FIG. 1B is a graph illustrating the enhanced stimulation indices of SC-β cells generated using various agents that specifically inhibit the level and/or activity of at least one ALK alone, or together in combination with additional agents that specifically inhibit the level and/or activity of at least one ALK and/or a sonic hedgehog signaling pathway inhibitor (e.g., SANT1), relative to the stimulation index of a control SC-β cell generated in the absence of using the indicated agents. Notably, combinations of agents tended to outperform individual agents. SB=SB431542; DM=DMH-1; and Alk=Alk5i.

FIG. 1C is a Table showing the parameters of the experiments performed to obtain the results shown in FIG. 1B.

FIG. 2A is a schematic illustrating six stages of differentiation of human pluripotent stem cells to SC-β cells. hPSC=human pluripotent stem cell, DE=definitive endoderm cell, GT=gut tube cell, PP1=pancreatic progenitor cell 1, PP2=pancreatic progenitor cell 2, EN=endocrine progenitor cell, SC-β=stem cell-derived β cells.

FIG. 2B is a schematic illustrating an exemplary six step differentiation protocol for generating SC-β cells from pluripotent stem cells, as described further in Pagliuca et al. 2014 and PCT International Application No. PCT/US2014/041992.

FIG. 2C is a schematic illustrating an exemplary method for generating SC-β cells according to the present invention, e.g., by adding an agent that specifically inhibits the level and/or activity of at least one ALK to Step 6 of the exemplary protocol shown in FIG. 2B.

FIG. 3 is a schematic illustration depicting an overview of the chemicals used and approach for the initial screen.

FIG. 4A shows graphs demonstrating improved in vivo function of SC-β cells generated using the agents and/or combinations of agents indicated (e.g., DMH1, Alk5i+SANT1, Alk5i+SB431542, and Alk5i+, SB431542+DMH1) 2 weeks post-transplantation of the SC-β cells into mice.

FIG. 4B is a Table quantifying the improvement in stimulation index and mean 30′ insulin post injection illustrated in FIG. 4A.

FIG. 5A shows graphs demonstrating improved in vivo function of SC-β cells generated using the agents and/or combinations of agents indicated (e.g., DMHI, Alk5i+SANT1, Alk5i+SB431542, and Alk5i+, SB431542+DMH1) 4 weeks post-transplantation of the SC-β cells into mice.

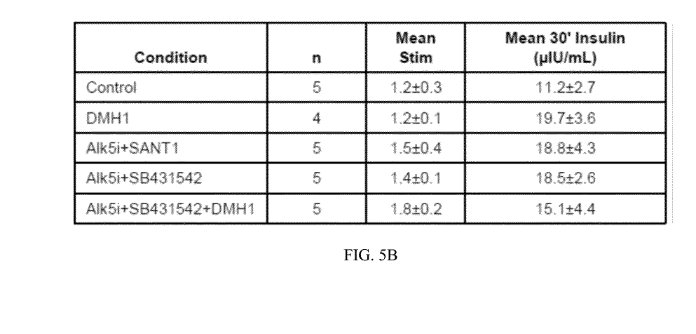

FIG. SB is a Table quantifying the improvement in stimulation index and mean 30′ insulin post injection illustrated in FIG. 5A.

### DETAILED DESCRIPTION OF THE INVENTION

The present invention is directed to generating SC-β cells, in particular SC-β cells that exhibit improved in vitro and in vivo function. More particularly, work described herein demonstrates that SC-β cells generated by endocrine progenitor cells directed to differentiate into SC-β cells with an agent that specifically inhibits the level and/or activity of at least one activin receptor-like kinase (e.g., ALK inhibitor) exhibit a greater stimulation index relative to SC-β cells generated using the same protocol but in the absence of contact with the ALK inhibitor.

The inventors screened compounds (including ALK inhibitors, MEK inhibitors, and MAPK inhibitors listed in Table 1 below) for their effect on in vitro SC-β cell function, and surprisingly and unexpectedly demonstrated that whereas agents that specifically inhibited at least one ALK improved the stimulation index of the resulting SC-β cells, agents that inhibited MAPK and/or MEK worsened the stimulation index of resulting SC-β cells.

TABLE 1
Compounds screened for effect on in vitro SC-β cell function

| Compound | Inhibits | Effect on Beta Cells |
|---|---|---|
| U0126 | MEK1, MEK2 | Reduced apoptosis in beta cells |
| SB203580 | p38 MAPK | Reduced apoptosis in beta cells |
| SB202190 | p38 MAPK | Reduced apoptosis in beta cells |
| | | May activate pathway that increases |
| | | insulin secretion in beta cells |
| SD208 | ALK5 | Helps maintain growth of human |
| | | pancreatic cancer cells |
| A83-01 | ALK1-7, | Reduces apoptosis of pancreatic beta cells, |
| | MAPK | increases insulin secretion |
| SB431542 | ALK4, ALK5, | Reduces apoptosis of pancreatic beta cells, |
| | ALK7 | increases insulin secretion |
| DMH-1 | ALK2 | Aids differentiation of zebrafish beta cells |

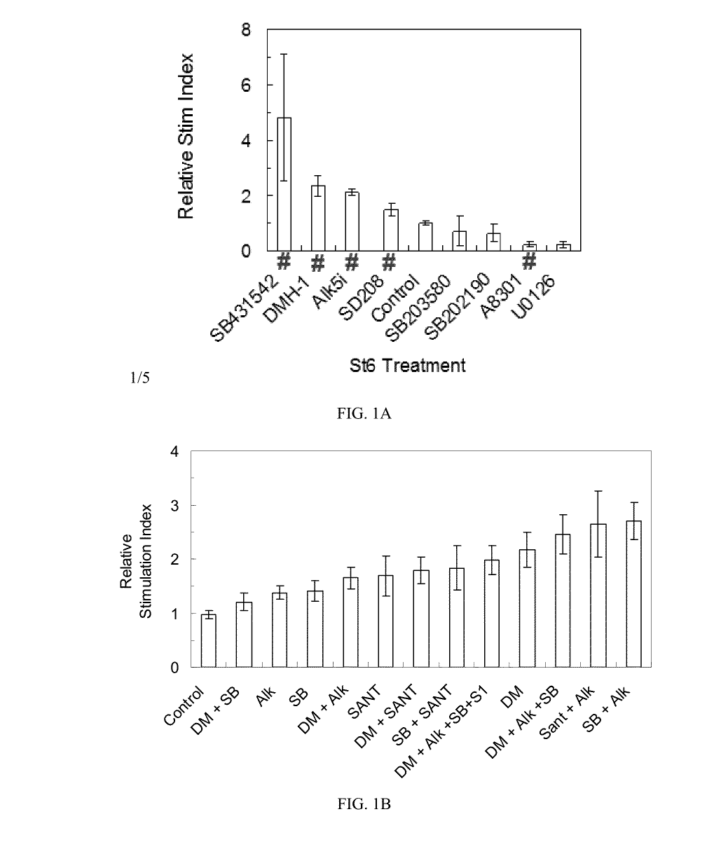

In particular, as shown in FIG. 1A, SB431542 (1 μM), DMH-1 (1 μM), Alk5i (110 μM), and SD208 (1 μM) each specifically inhibit at least one ALK and are not known to significantly inhibit MAPK, whereas A8301 is both an ALK inhibitor and a MAPK inhibitor, and groups with other MAPK inhibitors and/or MEK inhibitors, such as SB203580, SB202190 and U0126, which resulted in SC-β cells exhibiting worse stimulation indices than control SC-β cells.

Work described herein further demonstrates that the stimulation indices of SC-β cells can be improved compared to the stimulation indices of control SC-β cells by employing various combinations of agents that specifically inhibit the level and/or activity of at least one ALK alone, or together in combination with a sonic hedgehog signaling pathway inhibitor (e.g., SANT1), as is shown in FIG. 1B and FIG. 1C.

Some Definitions

“Differentiation” is the process by which an unspecialized (“uncommitted”) or less specialized cell acquires the features of a specialized cell such as, for example, a pancreatic cell. A differentiated cell is one that has taken on a more specialized (“committed”) position within the lineage of a cell. The term “committed”, when applied to the process of differentiation, refers to a cell that has proceeded in the differentiation pathway to a point where, under normal circumstances, it will continue to differentiate into a specific cell type or subset of cell types, and cannot, under normal circumstances, differentiate into a different cell type or revert to a less differentiated cell type. As used herein, the lineage of a cell defines the heredity of the cell, i.e., which cells it came from and to what cells it can give rise. The lineage of a cell places the cell within a hereditary scheme of development and differentiation. A lineage-specific marker refers to a characteristic specifically associated with the phenotype of cells of a lineage of interest and can be used to assess the differentiation of an uncommitted cell to the lineage of interest.

As used herein, “markers”, are nucleic acid or polypeptide molecules that are differentially expressed in a cell of interest. Differential expression means an increased level for a positive marker and a decreased level for a negative marker as compared to an undifferentiated cell. The detectable level of the marker nucleic acid or polypeptide is sufficiently higher or lower in the cells of interest compared to other cells, such that the cell of interest can be identified and distinguished from other cells using any of a variety of methods known in the art.

As used herein, a cell is “positive” or “+” for a specific marker (e.g., expresses the marker) when the specific marker is sufficiently detected in the cell. Similarly, the cell is “negative” or “−” for a specific marker when the specific marker is not sufficiently detected in the cell. For example, positive by FACS is usually greater than 2%, whereas the negative threshold by FACS is usually less than 1%.

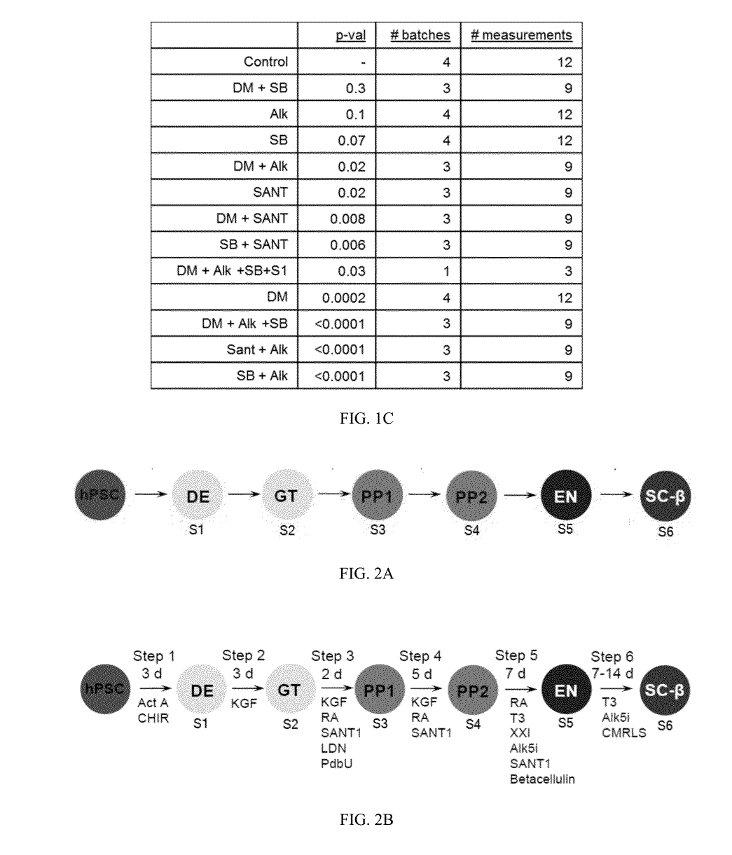

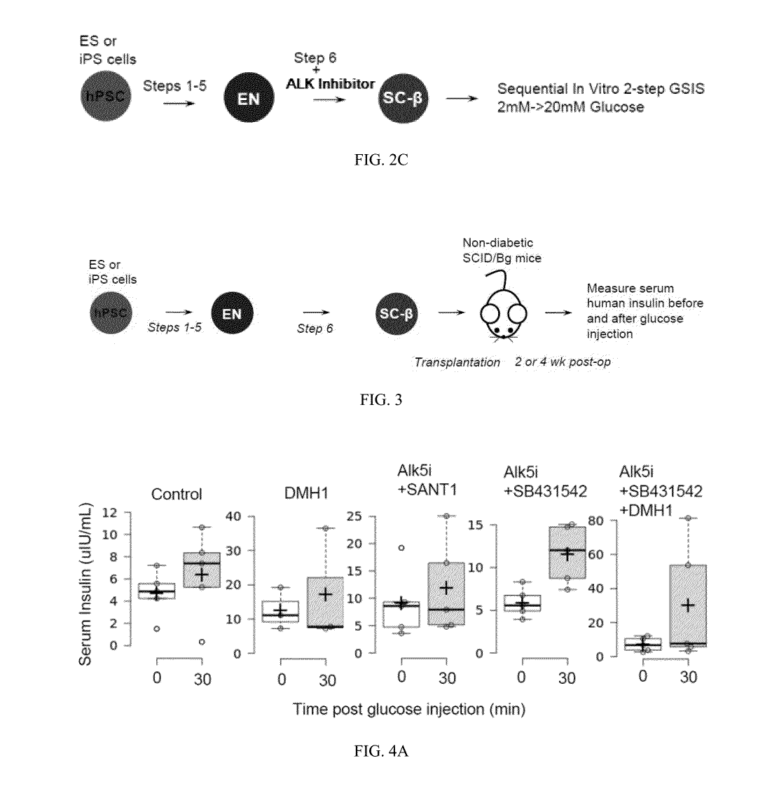

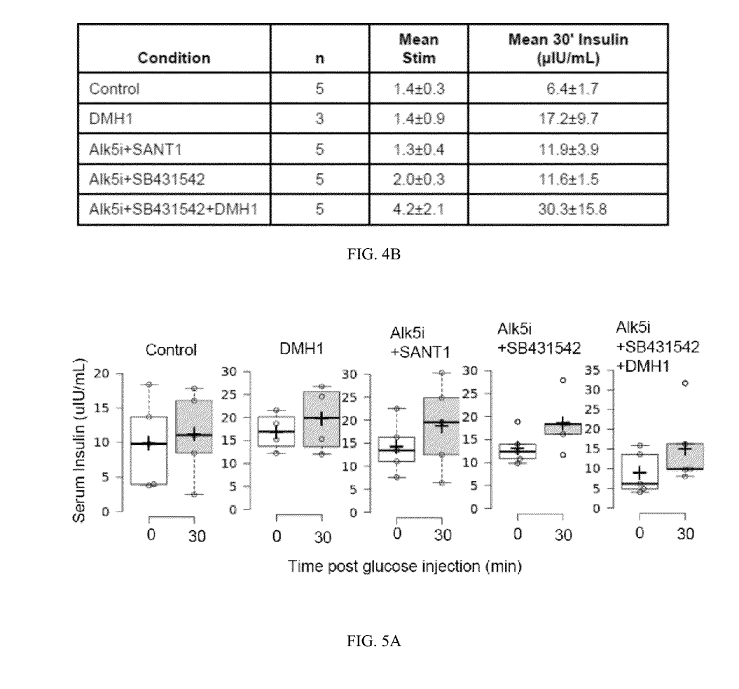

The process of differentiating pluripotent stem cells into functional pancreatic endocrine cells (i.e., SC-β cells) in vitro may be viewed as progressing through six consecutive stages, as is shown in the exemplary protocol depicted in FIG. 2A. In this step-wise progression, “Stage 1” or “S1” refers to the first step in the differentiation process, the differentiation of pluripotent stem cells into cells expressing markers characteristic of definitive endoderm cells (“DE”, “Stage 1 cells” or “S1 cells”). “Stage 2” refers to the second step, the differentiation of cells expressing markers characteristic of definitive endoderm cells into cells expressing markers characteristic of gut tube cells (“GT”, “Stage 2 cells” or “S2 cells”). “Stage 3” refers to the third step, the differentiation of cells expressing markers characteristic of gut tube cells into cells expressing markers characteristic of pancreatic progenitor 1 cells (“PP1”, “Stage 3 cells” or “S3 cells”). “Stage 4” refers to the fourth step, the differentiation of cells expressing markers characteristic of pancreatic progenitor 1 cells into cells expressing markers characteristic of pancreatic progenitor 2 cells (“PP2”, “Stage 4 cells” or “S4 cells”). “Stage 5” refers to the fifth step, the differentiation of cells expressing markers characteristic of pancreatic progenitor 2 cells into cells expressing markers characteristic of pancreatic endoderm cells and/or pancreatic endocrine progenitor cells (“EN”, “Stage 5 cells” or “S5 cells”). “Stage 6” refers to the differentiation of cells expressing markers characteristic of pancreatic endocrine progenitor cells into cells expressing markers characteristic of pancreatic endocrine β cells (“SC-β cells”, “Stage 6 cells” or “S6 cells”). It should be appreciated, however, that not all cells in a particular population progress through these stages at the same rate, i.e., some cells may have progressed less, or more, down the differentiation pathway than the majority of cells present in the population.

Characteristics of the various cell types associated with the stages shown in FIG. 2A are now described. “Definitive endoderm cells,” as used herein, refers to cells which bear the characteristics of cells arising from the epiblast during gastrulation and which form the gastrointestinal tract and its derivatives. Definitive endoderm cells express at least one of the following markers: FOXA2 (also known as hepatocyte nuclear factor 3β (“HNF3β”)), GATA4, SOX17, CXCR4, Brachyury, Cerberus, OTX2, goosecoid, C-Kit, CD99, and MIXL1. Markers characteristic of the definitive endoderm cells include CXCR4, FOXA2 and SOX17. Thus, definitive endoderm cells may be characterized by their expression of CXCR4, FOXA2 and SOX17. In addition, depending on the length of time cells are allowed to remain in Stage 1, an increase in HNF4α may be observed.

“Gut tube cells,” as used herein, refers to cells derived from definitive endoderm that can give rise to all endodermal organs, such as lungs, liver, pancreas, stomach, and intestine. Gut tube cells may be characterized by their substantially increased expression of HNF4α over that expressed by definitive endoderm cells. For example, a ten to forty fold increase in mRNA expression of HNF4α may be observed during Stage 2.

“Pancreatic progenitor 1 cells,” as used herein, refers to endoderm cells that give rise to the esophagus, lungs, stomach, liver, pancreas, gall bladder, and a portion of the duodenum. Pancreatic progenitor 1 cells express at least one of the following markers: PDX1, FOXA2, CDX2, SOX2, and HNF4α. Pancreatic progenitor 1 cells may be characterized by an increase in expression of PDX1, compared to gut tube cells. For example, greater than fifty percent of the cells in Stage 3 cultures typically express PDX1.

“Pancreatic progenitor 2 cells,” as used herein, refers to cells that express at least one of the following markers: PDX1, NKX6.1, HNF6, NGN3, SOX9, PAX4, PAX6, ISL1, gastrin, FOXA2, PTF1a, PROX1 and HNF4α. Pancreatic progenitor 2 cells may be characterized as positive for the expression of PDX1, NKX6.1, and SOX9.

“Pancreatic endocrine progenitor cells” or “endocrine progenitor cells” are used interchangeably herein to refer to pancreatic endoderm cells capable of becoming a pancreatic hormone expressing cell. Pancreatic endocrine progenitor cells express at least one of the following markers: NGN3; NKX2.2; NeuroD1; ISL1; PAX4; PAX6; or ARX. Pancreatic endocrine progenitor cells may be characterized by their expression of NKX2.2 and NeuroD1.

A “precursor thereof” as the term relates to a pancreatic endocrine progenitor cell refers to any cell that is capable of differentiating into a pancreatic endocrine progenitor cell, including for example, a pluripotent stem cell, a definitive endoderm cell, a gut tube cell, or a pancreatic progenitor cell, when cultured under conditions suitable for differentiating the precursor cell into the pancreatic pro endocrine cell.

“Pancreatic endocrine cells,” as used herein, refer to cells capable of expressing at least one of the following hormones: insulin, glucagon, somatostatin, ghrelin, and pancreatic polypeptide. In addition to these hormones, markers characteristic of pancreatic endocrine cells include one or more of NGN3, NeuroD1, ISL1, PDX1, NKX6.1, PAX4, ARX, NKX2.2, and PAX6. Pancreatic endocrine cells expressing markers characteristic of β cells can be characterized by their expression of insulin and at least one of the following transcription factors: PDX1, NKX2.2, NKX6.1, NeuroD1, ISL1, HNF30, MAFA and PAX6.

The terms “stem cell-derived β cell” and “SC-β cell” are used interchangeably herein to refer to cells differentiated in vitro (e.g., from pluripotent stem cells) that display at least one marker indicative of a pancreatic β cell (e.g., PDX-1 or NKX6-1), expresses insulin, and display a GSIS response characteristic of an endogenous mature β cell both in vitro and in vivo. The GSIS response of the SC-β cells can be observed within two weeks of transplantation of the SC-β cell into a host (e.g., a human or animal). It is to be understood that SC-β cells need not be derived (e.g., directly) from stem cells, as the methods of the disclosure are capable of deriving SC-β cells from any endocrine progenitor cell that expresses insulin or precursor thereof using any cell as a starting point (e.g., one can use embryonic stem cells, induced-pluripotent stem cells, progenitor cells, partially reprogrammed somatic cells (e.g., a somatic cell which has been partially reprogrammed to an intermediate state between an induced pluripotent stem cell and the somatic cell from which it was derived), multipotent cells, totipotent cells, a transdifferentiated version of any of the foregoing cells, etc, as the invention is not intended to be limited in this manner). In some aspects, human cells are excluded that are derived from human embryonic stem cells obtained exclusively by a method necessitating the destruction of an embryo. The skilled artisan is well aware of such methods and how to avoid them for the purposes of generating SC-β cells according to the methods of the present invention.

Used interchangeably herein are “d1”, “1d”, and “day 1”; “d2”, “2d”, and “day 2”, etc. These number letter combinations refer to a specific day of incubation in the different stages during the stepwise differentiation protocol of the instant application.

Methods for Generating SC-β Cells

FIG. 2C is a schematic depicting an overview of an exemplary method for generating SC-β cells in accordance with the present invention. In accordance with an example embodiment of the present invention, a method for generating stem cell-derived β (SC-β) cells comprises contacting a cell population comprising endocrine progenitor cells directed to differentiate into SC-β cells, or cell precursors thereof, with an effective amount of an agent (e.g., a first agent) that specifically inhibits the level and/or activity of at least one activin receptor-like kinase (ALK), thereby generating SC-β cells.

“Contacting”, “contacting the cell” and any derivations thereof as used herein, refers to any means of introducing an agent (e.g., nucleic acids, peptides, ribozymes, antibodies, small molecules, etc.) into a target cell or an environment in which the cell is present (e.g., cell culture), including chemical and physical means, whether directly or indirectly. Contacting also is intended to encompass methods of exposing a cell, delivering to a cell, or ‘loading’ a cell with an agent by viral or non-viral vectors, and wherein such agent is bioactive upon delivery. The method of delivery will be chosen for the particular agent and use. Parameters that affect delivery, as is known in the medical art, can include, inter alia, the cell type affected, and cellular location. In some aspects, contacting includes administering the agent to a subject. In some aspects, contacting refers to exposing a cell or an environment in which the cell is located (e.g., cell culture medium) to the agent that specifically inhibits the level and/or activity of at least one ALK.

In accordance with aspects of the present invention, the method further includes contacting the cell population with an effective amount of at least a second agent that specifically inhibits the level and/or activity of at least one ALK. In accordance with aspects of the present invention, the method further includes contacting the cell population with an effective amount of at least a second agent that specifically inhibits the level and/or activity of at least one ALK, and/or at least a third agent that specifically inhibits the level and/or activity of at least one ALK. The first agent, the second agent, and/or third agent may each specifically inhibit the level and/or activity of the same at least one ALK, or different at least one ALKs. The first agent, the second agent, and/or third agent may specifically inhibit the level and/or activity of overlapping ALKs, e.g., the first agent could inhibit at least one ALK, at least two ALKs, at least three ALKs, or at least four ALKs, etc., and the second agent and/or the third agent could inhibit one or more of those ALKs. In some aspects, each of the first agent, the second agent, and/or the third agent specifically inhibits one ALK. In some aspects, each of the first agent, the second agent, and/or the third agent specifically inhibits two ALKs. In some aspects, each of the first agent, the second agent, and/or the third agent specifically inhibits three ALKs.

In some aspects, the endocrine progenitor cells comprise PDX1+/NKX6.1+/NEUROD1+/insulin+/glucagon−/somatostatin− cells.

It is believed that SC-β cells generated by contacting endocrine progenitor cells (or their precursors) directed to differentiate into SC-β cells according to any protocol will exhibit improved in vitro and in vivo function when contacted with an agent that specifically inhibits the level and/or activity of at least one ALK.

As used herein, “directed to differentiate” refers to the process of causing a cell of a first cell type to differentiate into a cell of a second cell type.

Recently, two protocols for directing the differentiation of pluripotent stem cells into insulin-producing endocrine cells that express key markers of mature pancreatic β cells (e.g., SC-β cells) have been reported, each of which includes differentiating cells into endocrine progenitor cells that can be directed to differentiate into SC-β cells, as well as protocols for directing the pancreatic endocrine progenitor cells into SC-β cells, which can be used in the method disclosed herein for generating SC-β cells. First, as shown in FIG. 2B, an exemplary six-stage protocol for the large-scale production of functional human β cells using human pluripotent stem cells (hPSC) by sequential modulation of multiple signaling pathways in a three-dimensional cell culture system, without using any transgenes or genetic modification, was used to generate glucose-responsive, monohormonal insulin-producing cells that exhibited key β cell markers and β cell ultrastructure (see Pagliuca et al., 2014 and PCT International Application No. PCT/US2014/041992, both of which are incorporated herein by reference in their entirety). Pagliuca and colleagues reported that such cells mimicked the function of human islets in vitro and in vivo, and demonstrated the potential utility of such cells for in vivo transplantation to treat diabetes. Secondly, a seven-stage protocol that converts human embryonic stem cells (hESCs) into insulin-producing cells that expressed key markers of mature pancreatic β cells, such as MAFA, and displayed glucose-stimulated insulin secretion like that of human islets using static incubations in vitro was described (Rezania et al., 2014). Cells produced by such protocol, referred to as S7 cells, were found to rapidly reverse diabetics in mice within a little over a month.

In some aspects, the endocrine progenitor cells are directed to differentiate into SC-β cells by contacting the endocrine progenitor cells under conditions that promote cell clustering with i) a transforming growth factor β (TGF-β) signaling pathway inhibitor and ii) a thyroid hormone signaling pathway activator to induce the in vitro maturation of at least some of the endocrine progenitor cells into SC-β cells. In some aspects, the endocrine progenitor cells are optionally contacted with a protein kinase inhibitor (e.g., staurosporine).

In some aspects, the cell precursors are selected from the group consisting of pluripotent stem cells, SOX17+ definitive endoderm cells, PDX1+ primitive gut tube cells, PDX1+/NKX6.1+ pancreatic progenitor cells, PDX1+/NKX6.1+/NEUROD1+ endocrine progenitor cells, and combinations thereof.

The methods of the present invention contemplate contacting cells (e.g., endocrine progenitor cells or precursors thereof) with effective amounts of one or more agents that specifically inhibits the level and/or activity of at least one ALK. An “effective amount” of an agent (or composition containing such agent) refers to the amount sufficient to achieve a desired effect, e.g., when delivered to a cell or subject according to a selected administration form, route, and/or schedule. As will be appreciated by those of ordinary skill in this art, the absolute amount of a particular agent or composition that is effective may vary depending on such factors as the desired biological or pharmacological endpoint, the agent to be delivered, the target tissue, etc. Those of ordinary skill in the art will further understand that an “effective amount” may be contacted with cells or administered in a single dose, or the desired effect may be achieved by use of multiple doses. An effective amount of a composition may be an amount sufficient to reduce the severity of or prevent one or more symptoms or signs of a disorder (e.g., diabetes). In some aspects, the effective amount of the agent that specifically inhibits the level and/or activity of at least one ALK comprises a concentration of between about 0.1 μM and about 110 μM. In some aspects, the effective amount of the agent comprises 1 μM. In some aspects, the effective amount of the agent comprises 2 μM. In some aspects, the effective amount of the agent comprises 3 μM. In some aspects, the effective amount of the agent comprises 4 μM. In some aspects, the effective amount of the agent comprises 5 μM. In some aspects, the effective amount of the agent comprises 6 μM. In some aspects, the effective amount of the agent comprises 7 μM. In some aspects, the effective amount of the agent comprises 8 μM. In some aspects, the effective amount of the agent comprises 9 μM. In some aspects, the effective amount of the agent comprises 10 μM. In some aspects, the effective amount of the agent comprises 110 μM. In some aspects, the endocrine progenitor cells (S5 cells) are contacted with 1 μM of DMH1 to generate SC-β cells exhibiting an improved in vitro and/or in vivo function. In some aspects, the endocrine progenitor cells (S5 cells) are contacted with 1 μM of SB431542 to generate SC-β cells exhibiting an improved in vitro and/or in vivo function. In some aspects, the endocrine progenitor cells (S5 cells) are contacted with 110 μM of Alk5i to generate SC-β cells exhibiting an improved in vitro and/or in vivo function. In some aspects, the endocrine progenitor cells (S5 cells) are not contacted with Alk5i at a concentration of other than 10 μM to generate SC-β cells exhibiting an improved in vitro and/or in vivo function. In some aspects, the endocrine progenitor cells (S5 cells) are contacted with 1 μM of SD208 to generate SC-β cells exhibiting an improved in vitro and/or in vivo function. In some aspects, the endocrine progenitor cells (S5 cells) are contacted with 1 μM of DMH1 to generate SC-β cells exhibiting an improved in vitro and/or in vivo function. In some aspects, the endocrine progenitor cells (S5 cells) are contacted with 1 μM of DMH1 and 1 μM of SB431542 to generate SC-β cells exhibiting an improved in vitro and/or in vivo function. In some aspects, the endocrine progenitor cells (S5 cells) are contacted with 1 μM of DMH1 and 110 μM of Alk5i to generate SC-β cells exhibiting an improved in vitro and/or in vivo function. In some aspects, the endocrine progenitor cells (S5 cells) are contacted with 1 μM of DMH1, 1 μM of SB431542, and 110 μM of Alk5i to generate SC-β cells exhibiting an improved in vitro and/or in vivo function. In accordance with aspects of the invention, the effective amount of the second agent comprises a concentration range of between 0.1 μM and 110 μM. In accordance with aspects of the invention, the effective amount of the third agent comprises a concentration range of between 0.1 μM and 110 μM.

Activin Receptor-Like Kinase (ALK)

Activin receptor-like kinases are a subclass of cell-surface receptors exhibiting transmembrane protein serine/threonine kinase activity. Activins are dimeric growth and differentiation factors belonging to the transforming growth factor-beta (TGFβ) superfamily of structurally similar signaling proteins. Activins signal through a heteromeric complex of receptor serine kinases which include at least two type I (I and IB) and two type II (II and IIB) receptors, which are all transmembrane proteins made of a ligand-binding extracellular domain having a cysteine-rich region, a transmembrane domain, and a cytoplasmic domain having predicted serine/threonine specificity. Type receptors are important for signaling, whereas type II receptors are needed for binding ligands and expressing type I receptors. Type I and II receptors form stable complexes when ligands bind leading to phosphorylation of type I receptors by type II receptors. Activin signaling via ALK receptors has been further reviewed (see, e.g., Tsuchida et al. 2009). There are seven known ALK receptors, including ALK1 (Gene ID: 94; also known as HHT, ACVRL1, HHT2, ORW2, SKR3, ALK-1, TSR-1, and ACVRLK1), ALK2 (Gene ID: 90; also known as FOP; SKR1; TSRI; ACTRI; ACVR1A; and ACVRLK2), ALK3 (Gene ID: 657; also known as BMPR1A, SKR5; CD292; ACVRLK3; 10q23del), ALK4 (Gene ID: 91; also known as ACVR1B, SKR2; ACTRIB; and ACVRLK4), ALK5 (Gene ID: 7046; also known as TGFBR1; AAT5; ESS1; LDS1; MSSE; SKR4; ALK-5; LDS1A; LDS2A; TGFR-1; ACVRLK4; and tbetaR-I), ALK6 (Gene ID: 658; also known as BMPR1B; ALK-6; and CDw293), and ALK7 (Gene ID: 130399; also known as ACVR1C and ACVRLK7).

The present invention contemplates using any agent that specifically inhibits the level and/or activity of at least one ALK (also referred to herein as a “ALK inhibitor”) in the method for generating SC-β cells.

ALK inhibitors can be small organic or inorganic molecules; saccharides; oligosaccharides; polysaccharides; biological macromolecules, e.g., peptides, proteins, and peptide analogs and derivatives; peptidomimetics; nucleic acids and nucleic acid analogs and derivatives (including but not limited to microRNAs, siRNAs, shRNAs, antisense RNAs, a ribozymes, and aptamers); an extract made from biological materials such as bacteria, plants, fungi, or animal cells; animal tissues; naturally occurring or synthetic compositions; and any combinations thereof.

In accordance with aspects of the present invention, the at least one ALK is selected from the group consisting of ALK1, ALK2, ALK3, ALK4, ALK5, ALK6 and ALK7.

ALK1 Inhibitors

In accordance with aspects of the present invention, the at least one ALK comprises ALK1, i.e., the first agent, second agent, and/or third agent that specifically inhibits at least one ALK specifically inhibits at least ALK1.

Exemplary ALK1 inhibitors include, but are not limited to, small molecule inhibitors such as dorsomorphin, LDN-193189, a hydroxymethylaryl-substituted pyrrolotriazine ALK inhibitor described in U.S. Pub. No. 2014/0256718, ML347, and K02288, a biologic inhibitor such as PF-3446962, a fully human monoclonal antibody against ALK1, ALK1-Fc fusion protein (amino acids 23-119 of mouse ALK1), and ACE-041, a human ALK1-Fc fusion protein as described in U.S. Pub. No. 2014/0193425, a humanized or fully human antibody that binds to an ALK1 ligand described in U.S. Pub, No. 2014/0227254, e.g., a humanized form of MAB3209, an ALK1 extracellular (ECD)-Fc fusion protein described in U.S. Pat. No. 8,455,428, an antibody or antibody fragment specifically binding ALK1 and/or an antibody or antibody fragment specifically binding an ALK1 ligand, an endoglin ECD antibody, an endoglin ECD, a BMP9 pro-peptide, and a BMP10 pro-peptide each of which is described in detail in WIPO Pub. No. WO/2014/055869, and a human monocolonal antibody that binds to the ECD of ALK-1 described in U.S. Pub. No. 2010/0197005.

ALK2 Inhibitors

In accordance with aspects of the present invention, the at least one ALK comprises ALK2, i.e., the first agent, second agent, and/or third agent that specifically inhibits at least one ALK specifically inhibits at least ALK2.

Exemplary ALK2 inhibitors include compounds of Formula I and stereoisomers, pharmaceutically acceptable salts, tautomers, or prodrugs thereof:

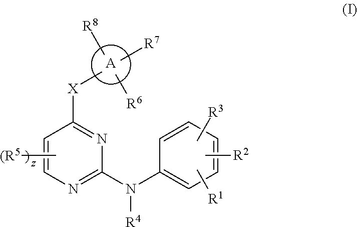

wherein

A represents a 6-membered aromatic ring or a 5 or 6-membered heteroaryl ring;

X is —NH—, —O—, —S(O)ₘ—, —CH₂—, —CHOH— or —C(═O)—;

R¹ is H, halo, C₁-C₆ alkyl, C₂-C₆ alkenyl, C₂-C₆ alkynyl, C₁-C₆ haloalkyl, C C₆ alkoxy, —S(O)ₘ C C₆ alkyl, C C₆ hydroxylalkyl, —OCH₂CH₂R⁹, —(CH₂)ₙNR^{a}R^{b}, or —CONR^{a}R^{b};

R² is halo, C₁-C₆ alkyl, C₂-C₆ alkenyl, C₂-C₆ alkynyl, C₁-C₆ haloalkyl,

C₁-C₆ alkoxy, —S(O)ₘ C₁-C₆ alkyl, C₁-C₆ hydroxylalkyl, —OCH₂CH₂R⁹, —(CH₂)ₙNR^{a}R^{b}, or —CONR^{a}R^{b};

R³ is halo, C₁-C₆ alkyl, C₂-C₆ alkenyl, C₂-C₆ alkynyl, C₁-C₆ haloalkyl, C₁-C₆ alkoxy, —S(O)ₘ C₁-C₆ alkyl, C₁-C₆ hydroxylalkyl, —OCH₂CH₂R⁹, —(CH₂)ₙNR^{a}R^{b}, —CONR^{a}R^{b} or —NHCHR^{a}R^{b};

R⁴ is H or C₁-C₆ alkyl;

R⁵ is, at each occurrence, independently H, halo, C₁-C₆ alkyl, C₁-C₆ alkoxy, C₃-C₆ cycloalkoxy, —CN, C₁-C₆ nitrilylalkyl or C₃-C₆ nitrilylcycloalkyl;

R⁶ and R⁷ are each independently H, halo, hydroxyl, C₁-C₆ alkyl, C₁-C₆ alkoxy, C₃-C₆ cycloalkoxy, C₁-C₆ nitrilylalkyl, C₃-C₆ nitrilylcycloalkyl, C₃-C₆ nitrilylcycloalkylalkyl or —(CH₂)ₙNR^{a}R^{b}; R⁸ is H, halo, hydroxyl, C₁-C₆ alkyl, C₁-C₆ alkoxy, C3-C6 cycloalkoxy, C₁-C₆ nitrilylalkyl, C₃-C₆ nitrilylcycloalkyl, C₃-C₆ nitrilylcycloalkylalkyl, (CH₂)ₙNR^{a}R^{b}, aryl or heteroaryl;

R⁹ is —H, —Cl, C₁-C₄ alkyl, C₂-C₃ alkenyl, C₂-C₃ alkynyl, C₃-C₄ cycloalkyl, —CH₂OH, —OCH₃, —OCH₂CH₃, —S(O)ₘCH₃, —CH₂CN, —CH₂OCH₃, —CH₂S(O)ₘCH₃, —CN, —CHCH₃CN, —C(CH₃)₂CN or

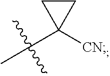 [SMILES: *C1(C#N)CC1]

R^{a} and R^{b} are each independently —H, C₁-C₆ alkyl, C₁-C₆ hydroxylakly, or R^{a} and R^{b} together with the nitrogen or carbon atom to which they are attached form an optionally substituted 5 or 6 membered saturated carbocyclic or heterocyclic ring;

m is 0, 1 or 2; and

n is 0, 1, 2 or 3. Compounds of formula I are described in further detail in WIPO Pub. WO2014/151871.

Exemplary ALK2 inhibitors also include dorsomorphin, which selectively blocks ALK2, ALK3 and ALK6 activity, LDN-193189, which inhibits transcriptional activity of the BMP type I receptors ALK2 and ALK3 (IC50=5 nM and 30 nM, respectively), LDN-212854, ML347, and LDN-193189 HCL, and K02288.

In accordance with aspects of the present invention, the first agent, second agent, and/or third agent that specifically inhibits at least ALK2 comprises 4-[6-[4-(1-Methylethoxy)phenyl]pyrazolo[1,5-a]pyrimidin-3-yl]-quinoline(DMH-1 or DMH1).

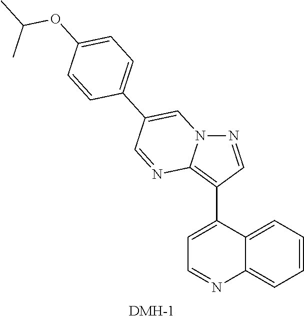 [SMILES: CC(C)Oc1ccc(-c2cnc3c(-c4ccnc5ccccc45)cnn3c2)cc1]

DMH-1 is a selective inhibitor of the bone morphogenic protein (BMP) ALK2 receptor (IC₅₀=107.9 nM), which exhibits no detectable inhibition of AMPK, ALK5, KDR (VEGFR-2) or PDGFRβ receptors. DMH-1 has been shown to block BMP4-induced phosphorylation of Smads 1, 5 and 8 in HEK293 cells.

ALK3 Inhibitors

In accordance with aspects of the present invention, the at least one ALK comprises ALK3, i.e., the first agent, second agent, and/or third agent that specifically inhibits at least one ALK specifically inhibits at least ALK3.

Exemplary ALK3 inhibitors include, but are not limited to, LDN-193189, and LDN-193189 HCL.

ALK4 Inhibitors

In accordance with aspects of the present invention, the at least one ALK comprises ALK4, i.e., the first agent, second agent, and/or third agent that specifically inhibits at least one ALK specifically inhibits at least ALK4.

Exemplary ALK4 inhibitors include, but are not limited to, SB525334, EW-7197, and SB505124.

In accordance with aspects of the present invention, the first agent, second agent, and/or third agent that specifically inhibits at least ALK4 comprises 4-[4-(1,3-benzodioxol-5-yl)-5-(2-pyridinyl)-1H-imidazol-2-yl]benzamide (SB431542).

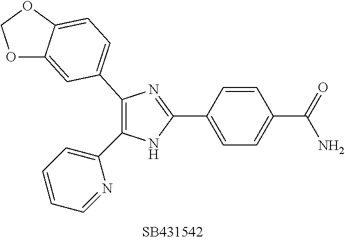 [SMILES: NC(=O)c1ccc(-c2nc(-c3ccc4c(c3)OCO4)c(-c3ccccn3)[nH]2)cc1]

The IC50 value of SB431542 for ALK4 is 140 nM.

ALK5 Inhibitors

In accordance with aspects of the present invention, the at least one ALK comprises ALK5, i.e., the first agent, second agent, and/or third agent that specifically inhibits at least one ALK specifically inhibits at least ALK5.

Exemplary ALK5 inhibitors include, but are not limited to 2-(5-Chloro-2-fluorophenyl) pteridin-4-yl]pyridin-4-yl-amine (SD208), LY2109761, SB525334, EW-7197, and HTS466284.

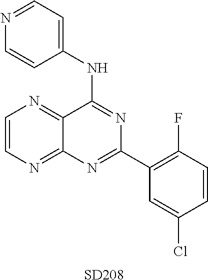 [SMILES: Fc1ccc(Cl)cc1-c1nc(Nc2ccncc2)c2nccnc2n1 ; "SD208"]

SD-208 is TGF-βR I kinase inhibitor with IC₅₀=49 nM based on direct enzymatic assay of TGFRI kinase (ALK5) activity with a specificity of >100-fold against TGFRII and at least 17-fold over members of a panel of related protein kinases including p38a, p38b, p38d, JNK1, EGFR, MAPKAPK2, MKK6, ERK2, PKC, PKA, PKD, CDC2, and CaMKII.

In accordance with aspects of the present invention, the first agent, second agent, and/or third agent that specifically inhibits at least ALK5 comprises SB431542. SB43152 is also potent and selective inhibitor of ALK5 with IC50 of 94 nM, exhibiting 100-fold more selectivity for ALK5 than p38 MAPK and other kinases.

In accordance with aspects of the present invention, the first agent, second agent, and/or third agent specifically inhibits ALK5 without detectably inhibiting MAPK kinases such as p38 MAPK. In accordance with aspects of the present invention, the first agent, second agent, and/or third agent specifically inhibits ALK5 with at least 100-fold more selectivity for ALK5 than p38 MAPK.

In accordance with aspects of the present invention, the first agent, second agent, and/or third agent that specifically inhibits at least ALK5 comprises Alk5 inhibitor II (also referred to herein as Alk5i).

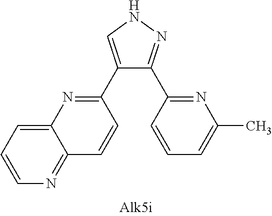 [SMILES: Cc1cccc(-c2n[nH]cc2-c2ccc3ncccc3n2)n1]

Alk5i is a cell permeable, potent, selective and ATP-competitive inhibitor of TGF-β RI kinase (IC₅₀=23 nM, 4 nM and 18 nM for binding, auto-phosphorylation and cellular assay in HepG2 cells of TGF-β RI kinase, respectively).

ALK6 Inhibitors

In accordance with aspects of the present invention, the at least one ALK comprises ALK6, i.e., the first agent, second agent, and/or third agent that specifically inhibits at least one ALK specifically inhibits at least ALK6.

Exemplary ALK6 inhibitors include dorsomorphin, K02288, and LDN193189.

In accordance with aspects of the present invention, the first agent, second agent, and/or third agent that specifically inhibits at least ALK6 comprises SB431542

ALK7 Inhibitors

In accordance with aspects of the present invention, the at least one ALK comprises ALK7, i.e., the first agent, second agent, and/or third agent that specifically inhibits at least one ALK specifically inhibits at least ALK7.

Exemplary ALK7 inhibitors include, but are not limited to SB-431542.

In accordance with aspects of the present invention, the at least one ALK is selected from the group consisting of ALK1, ALK2, ALK3, ALK4, ALK6 and ALK7.

In accordance with aspects of the present invention, the at least one ALK is other than ALK5.

In accordance with aspects of the present invention, the first agent, the second agent, and/or third agent specifically inhibits at least one ALK other than ALK5. In accordance with aspects of the present invention, the first agent, the second agent, and/or third agent specifically inhibits ALK5, and exhibits an IC₅₀ for MAPK that is greater than the IC50 of Alk5i for MAPK. In accordance with aspects of the present invention, the first agent, the second agent, and/or third agent specifically inhibits ALK5, specifically inhibits at least one ALK other than ALK5, and exhibits an IC₅₀ for MAPK that is equal to or greater than the IC50 of A8301 for MAPK. In accordance with aspects of the present invention, the first agent, the second agent, and/or third agent specifically inhibits ALK5, specifically inhibits at least one ALK other than ALK5, and exhibits an IC₅₀ for MAPK that is greater than the IC50 of Alk5i for MAPK.

In accordance with aspects of the present invention, the first agent, the second agent, and/or third agent exhibits an IC₅₀ for the at least one ALK that is less than or equal to 500 nm.

In accordance with aspects of the present invention, the cells are contacted with a concentration of the first agent, the second agent, and/or the third agent equal to or greater than its IC50 value for at least one ALK.

In accordance with aspects of the present invention, the first agent, the second agent, and/or the third agent is more selective (e.g., at least 10-fold, at least 20-fold, at least 30-fold, at least 40-fold, at least 50-fold, at least 60-fold, at least 70-fold, at least 80-fold, at least 90-fold, at least 100-fold, at least 200-fold, at least 300-fold, at least 400-fold, at least 500-fold, at least 1000-fold, at least 2000-fold, at least 3000-fold, at least 4000-fold, at least 5000-fold, or greater) for the at least one ALK than for at least one mitogen activated protein kinase (MAPK) or other kinases.

In accordance with aspects of the present invention, the first agent and/or the second agent is selected from the group consisting of SB431542, DMH-1, and Alk5 inhibitor II.

In accordance with aspects of the present invention, the at least one ALK is selected from the group consisting of SB431542 and DMH-1.

In some aspects, a ALK inhibitor decreases the level and/or activity of at least one ALK in cells contacted by at least 5%, 10%, 20%, 30%, 40%, 50%, 60%, 70%, 80%, 90%, 91%, 92%, 93%, 94%, 95%, 96%, 97%, 98%, 99%, or greater relative to the level or activity of the at least one ALK in the cells in the absence of contact with the ALK inhibitor. While not required, a ALK inhibitor can completely inhibit the level and/or activity of at least one ALK in the cells. It should be appreciated that the ALK inhibitors may decrease the level and/or activity of ALK in any cell in the population in which endocrine progenitor cells are differentiating into SC-β cells, including in the SC-β cells generated in the population, endocrine progenitor cells or any precursors thereof. As used herein, “level” refers to mRNA and/or protein expression levels of the at least one ALK. As used herein, “activity” includes activin activity, activin signaling through the at least one ALK, serine/threonine kinase activity, formation of stable complexes between type I and II receptors, phosphorylation of a type I receptor by a type II receptor, or phosphorylation of component in the activin signaling pathway.

It should be appreciated that the first agent, second agent, and/or third agent may act via any mechanism which results in decreased level and/or activity of ALK.

It should also be appreciated that the population of cells comprising the endocrine progenitor cells contacted in accordance with the method may comprise different cells types as the cells are differentiating into SC-β cells. Preferably, a maximum amount of endocrine cells in the population contacted with a ALK inhibitor differentiate into SC-β cells. In some aspects, between at least 5% and 65% of the endocrine cells in the population differentiate into SC-β cells.

SC-β Cells Obtained by the Method of Generating SC-β Cells

In accordance with an embodiment of the present invention, an isolated SC-β cell or population thereof generated according to a method described herein is provided. The isolated SC-β cell or population exhibits a GSIS response both in vitro and in vivo. The isolated SC-β cell or population also exhibits at least one characteristic feature of a mature endogenous β cell (e.g., monohormonality). In some aspects, an isolated SC-β cell or population thereof exhibits a stimulation index that is at least between 1.5-fold and 10-fold greater than the stimulation index of a control SC-β cell. In some aspects, an isolated SC-β cell or population thereof produces between approximately 300 uIU to about 4000 uIU per 30 minute per 10⁶ total cells incubation at a high glucose concentration. In some aspects, an isolated SC-β cell or population thereof two weeks after transplantation into a subject in vivo releases between 3 uIU/mL and 81 uIU/mL of insulin within 30 minutes of administering 2 g/kg glucose to the subject.

The SC-β cells disclosed herein share many distinguishing features of native β cells, but are different in certain aspects (e.g., gene expression profiles). In some embodiments, the SC-β cell is non-native. As used herein, “non-native” means that the SC-β cells are markedly different in certain aspects from β cells which exist in nature, i.e., native β cells. It should be appreciated, however, that these marked differences typically pertain to structural features which may result in the SC-β cells exhibiting certain functional differences, e.g., although the gene expression patterns of SC-β cells differs from native β cells, the SC-β cells behave in a similar manner to native β cells but certain functions may be altered (e.g., improved) compared to native β cells. For example, a higher frequency of SC-β cells respond to 20 mM glucose compared to the frequency of native β cells. Other differences between SC-β cells and native β cells would be apparent to the skilled artisan based on the data disclosed herein.

The SC-β, cells (e.g., human) generated according to the methods described herein may further exhibit at least one of the following characteristics of an endogenous mature pancreatic β cell: i) a response to multiple glucose challenges that resembles the response of endogenous islets (e.g., at least one, at least two, or at least three or more sequential glucose challenges); ii) a morphology that resembles the morphology of an endogenous β cell; iii) packaging of insulin into secretory granules or encapsulated crystalline insulin granules; iv) a stimulation index of greater than at least 1.4; v) cytokine-induced apoptosis in response to cytokines; vi) enhanced insulin secretion in response to known antidiabetic drugs (e.g., secretagogues); vii) monohormonal, i.e., they do not abnormally co-express other hormones, such as glucagon, somatostatin or pancreatic polypeptide; viii) a low rate of replication; and ix) increased intracellular Ca²⁺ in response to glucose.

In accordance with an embodiment of the present invention, a microcapsule comprising the isolated SC-β cell or population thereof encapsulated therein is provided.

In accordance with an embodiment of the present invention, a macroencapsulation device comprising the isolated SC-β cell or population thereof is provided.

In accordance with an embodiment of the present invention, a cell line comprising an isolated SC-β cell that stably expresses insulin is provided.

Assays

In accordance with an embodiment of the present invention, an isolated SC-β cell or population thereof generated according to the methods herein, or an SC-β cell that stably expresses insulin, can be used in various assays. In some aspects, an isolated SC-β cell, population thereof, or an SC-β cell that stably expresses insulin, can be used in an assay to identify one or more candidate agents which promote or inhibit a β cell fate selected from the group consisting of β cell proliferation, β cell replication, β cell death, β cell function, β cell susceptibility to immune attack, and β cell susceptibility to dedifferentiation or differentiation. In some aspects, an isolated SC-β cell, population thereof, or an SC-β cell that stably expresses insulin, can be used in an assay to identify one or more candidate agents which promote the differentiation of at least one insulin-positive endocrine cell or a precursor thereof into at least one SC-β cell. The assays typically involve contacting the isolated SC-β cell, population thereof, or an SC-β cell that stably expresses insulin, with one or more candidate agents to be assessed for its ability to i) promote or inhibit a β cell fate selected from the group consisting of β cell proliferation, β cell replication, β cell death, β cell function, β cell susceptibility to immune attack, and β cell susceptibility to dedifferentiation or differentiation, or ii) promoting the differentiation of at least one insulin-positive endocrine cell or a precursor thereof into at least one SC-β cell and assessing whether the candidate agent possesses the ability to i) promote or inhibit a β cell fate selected from the group consisting of β cell proliferation, β cell replication, β cell death, β cell function, β cell susceptibility to immune attack, and β cell susceptibility to dedifferentiation or differentiation, or ii) promoting the differentiation of at least one insulin-positive endocrine cell or a precursor thereof into at least one SC-β cell.

Methods for Treatment

In accordance with an embodiment of the present invention, methods for the treatment of a subject in need thereof are provided. The methods entail administering to a subject in need thereof an isolated population of SC-β cells, a microcapsule comprising SC-β cells encapsulated therein, and/or a macroencapsulation device comprising the SC-β cells encapsulated therein. In some aspects, the subject is in need of additional β cells. In some aspects, the subject has, or has an increased risk of developing diabetes. An SC-β cell or population (e.g., isolated) of SC-β cells generated by a method of the present invention can be administered to a subject for treatment of type 1 or type 2 diabetes. In some aspects, the subject has, or has an increased risk of developing, a metabolic disorder. In some aspects, administering to the subject comprises implanting SC-β cells, a microcapsule comprising SC-β cells, or a macroencapsulation device comprising SC-β cells into the subject. The subject may be a human subject or an animal subject. In some aspects, the cells may be implanted as dispersed cells or formed into clusters that may be infused into the hepatic portal vein. In some aspects, cells may be provided in biocompatible degradable polymeric supports, porous non-degradable devices or encapsulated to protect from host immune response. Cells may be implanted into an appropriate site in a recipient. The implantation sites include, for example, the liver, natural pancreas, renal subcapsular space, omentum, peritoneum, subserosal space, intestine, stomach, or a subcutaneous pocket.

To enhance further differentiation, survival or activity of the implanted cells in vivo, additional factors, such as growth factors, antioxidants or anti-inflammatory agents, can be administered before, simultaneously with, or after the administration of the cells. These factors can be secreted by endogenous cells and exposed to the administered cells in situ. Implanted cells can be induced to differentiate by any combination of endogenous and exogenously administered growth factors known in the art.

The amount of cells used in implantation depends on a number of various factors including the patient's condition and response to the therapy, and can be determined by one skilled in the art.

In some aspects, the method of treatment further comprises incorporating the cells into a three-dimensional support prior to implantation. The cells can be maintained in vitro on this support prior to implantation into the patient. Alternatively, the support containing the cells can be directly implanted in the patient without additional in vitro culturing. The support can optionally be incorporated with at least one pharmaceutical agent that facilitates the survival and function of the transplanted cells.

Artificial Islet or Pancreas

In accordance with an embodiment of the present invention, an artificial islet or pancreas is provided. The artificial islet or pancreas can be constructed using the SC-β cells generated according to the methods described herein.

An artificial pancreas is a device that encapsulates and nurtures islets of Langerhans to replace the islets and β cells destroyed by type I diabetes. An artificial pancreas may contain a million islets or more, and may be implanted in the peritoneal cavity or under the skin where it can respond to changing blood glucose levels by releasing hormones, such as insulin. An artificial pancreas may be made using living (e.g., glucose-sensing and insulin secreting islets) and nonliving components (e.g., to shield the islets from the diabetic's body and its destructive immune mechanism while permitting the islets to thrive).

The present invention contemplates using β cells in any artificial pancreas. In some aspects, the artificial pancreas comprises microencapsulated or coated islets comprising SC-β cells generated according to the methods herein. In some aspects, the artificial pancreas comprises a macroencapsulation device into which islet cells comprising SC-β cells generated according to the methods herein are grouped together and encapsulated. In some aspects, the macroencapsulation device comprises a PVA hydrogel sheet for an artificial pancreas of the present invention (Qi et al., 2004). In some aspects, the artificial islet comprises SC-β cells generated according to the methods herein, along with other islet cells (α, δ, etc.) in the form of an islet sheet. The islet sheet comprises a layer of artificial human islets comprising the SC-β cells macroencapsulated within a membrane (e.g., of ultra-pure alginate). The sheet membrane is reinforced with mesh and may be coated on the surface to prevent or minimize contact between the cells encapsulated inside and the transplantation recipient's host immune response. Oxygen, glucose, and other nutrients readily diffuse into the sheet through the membrane nurturing the islets, and hormones, such as insulin readily diffuse out. Additional examples of membranes designed for macroencapsulation/implantation of an artificial islet or pancreas can be found in the literature (Isayeva et al. 2003). Another example of a macroencapsulated implant suitable for an artificial islet or pancreas can be found in the literature (Aurélien, et al. 2014).

### TERMINOLOGY

The articles “a”, “an” and “the” as used herein, unless clearly indicated to the contrary, should be understood to include the plural referents. Claims or descriptions that include “or” between one or more members of a group are considered satisfied if one, more than one, or all of the group members are present in, employed in, or otherwise relevant to a given product or process unless indicated to the contrary or otherwise evident from the context. It should it be understood that, in general, where the invention, or aspects of the invention, is/are referred to as comprising particular elements, features, etc., certain embodiments of the invention or aspects of the invention consist, or consist essentially of, such elements, features, etc. For purposes of simplicity those embodiments have not in every case been specifically set forth in haec verba herein. It should also be understood that any embodiment of the invention, e.g., any embodiment found within the prior art, can be explicitly excluded from the claims, regardless of whether the specific exclusion is recited in the specification. For example, any ALK may be excluded from the genus of ALKs (e.g., ALK5), and any agent may be excluded from the subgenus of agents that specifically inhibit the at least one ALK (e.g., ALK5 inhibitors claimed (e.g., Alk5 inhibitor II)).

Where ranges are given herein, the invention includes embodiments in which the endpoints are included, embodiments in which both endpoints are excluded, and embodiments in which one endpoint is included and the other is excluded. It should be assumed that both endpoints are included unless indicated otherwise. Furthermore, it is to be understood that unless otherwise indicated or otherwise evident from the context and understanding of one of skill in the art, values that are expressed as ranges can assume any specific value or subrange within the stated ranges in different embodiments of the invention, to the tenth of the unit of the lower limit of the range, unless the context clearly dictates otherwise. It is also understood that where a series of numerical values is stated herein, the invention includes embodiments that relate analogously to any intervening value or range defined by any two values in the series, and that the lowest value may be taken as a minimum and the greatest value may be taken as a maximum. Numerical values, as used herein, include values expressed as percentages. For any embodiment of the invention in which a numerical value is prefaced by “about” or “approximately”, the invention includes an embodiment in which the exact value is recited. For any embodiment of the invention in which a numerical value is not prefaced by “about” or “approximately”, the invention includes an embodiment in which the value is prefaced by “about” or “approximately”. “Approximately” or “about” generally includes numbers that fall within a range of 1% or in some embodiments 5% of a number in either direction (greater than or less than the number) unless otherwise stated or otherwise evident from the context (except where such number would impermissibly exceed 100% of a possible value).

Furthermore, it is to be understood that the invention encompasses all variations, combinations, and permutations in which one or more limitations, elements, clauses, descriptive terms, etc., from one or more of the listed claims is introduced into another claim dependent on the same base claim (or, as relevant, any other claim) unless otherwise indicated or unless it would be evident to one of ordinary skill in the art that a contradiction or inconsistency would arise. Where elements are presented as lists, e.g., in Markush group or similar format, it is to be understood that each subgroup of the elements is also disclosed, and any element(s) can be removed from the group.

Certain claims are presented in dependent form for the sake of convenience, but any dependent claim may be rewritten in independent format to include the limitations of the independent claim and any other claim(s) on which such claim depends, and such rewritten claim is to be considered equivalent in all respects to the dependent claim (either amended or unamended) prior to being rewritten in independent format. It should also be understood that, unless clearly indicated to the contrary, in any methods claimed herein that include more than one act, the order of the acts of the method is not necessarily limited to the order in which the acts of the method are recited, but the invention includes embodiments in which the order is so limited. It is contemplated that all aspects described above are applicable to all different embodiments of the invention. It is also contemplated that any of the above embodiments can be freely combined with one or more other such embodiments whenever appropriate.

### REFERENCES

1. Bellin et al., (2012). Potent induction immunotherapy promotes long-term insulin independence after islet transplantation in type 1 diabetes. Am. J. Transplant. 12, 1576-1583.
2. Pagliuca et al. (2014). Generation of Functional Human Pancreatic β cells In Vitro. Cell. 159, 428-439.
3. Rezania et al. (2014). Reversal of diabetes with insulin-producing cells derived in vitro from human pluripotent stem cells. Nat. Biotech. 32(11), 1121-1133.
4. Isayeva, et al. (2003). Characterization and performance of membranes designed for macroencapsulation/implantation of pancreatic islet cells. Biomaterials 24(20), 3483-3491.
5. Motté, et al. (2014). Composition and function of macroencapsulated human embryonic stem cell-derived implants: comparison with clinical human islet cell grafts. American Journal of Physiology-Endocrinology and Metabolism 307(9), E838-E846.
6. Qi et al. (2004). PVA hydrogel sheet macroencapsulation of the bioartificial pancreas. Biomaterials 24(27), 5885-5892.
7. Tsuchida et al. (2009). Activin signaling as an emerging target for therapeutic interventions. Cell Communication & Signaling 7, 15.

What is claimed is:

1. A method for generating stem cell-derived β (SC-β) cells, the method comprising contacting a cell population comprising endocrine progenitor cells or precursors thereof under conditions suitable for directing differentiation of said cells into SC-β cells with an effective amount of a first agent that specifically inhibits the level and/or activity of at least one activin receptor-like kinase (ALK), thereby generating SC-β cells.

2. The method of claim 1, wherein the endocrine progenitor cells comprise PDX1+/NKX6.1+/NEUROD1+/insulin+/glucagon−/somatostatin− cells.

3. The method of claim 1, wherein the precursors are selected from the group consisting of pluripotent stem cells, SOX17+ definitive endoderm cells, PDX1+ primitive gut tube cells, PDX1+/NKX6.1+ pancreatic progenitor cells, PDX1+/NKX6.1+/NEUROD1+ endocrine progenitor cells.

4. The method of claim 1, wherein the endocrine progenitor cells are directed to differentiate into SC-β cells by contacting the endocrine progenitor cells under conditions that promote cell clustering with i) a transforming growth factor β (TGF-β) signaling pathway inhibitor and ii) a thyroid hormone signaling pathway activator to induce the in vitro maturation of at least some of the endocrine progenitor cells into SC-β cells.

5. The method of claim 1, wherein the effective amount of the first agent comprises a concentration range of between 0.1 μM and 110 μM.

6. The method of claim 1, further comprising contacting the cell population with an effective amount of at least a second agent that specifically inhibits the level and/or activity of at least one ALK.

7. The method of claim 6, wherein the effective amount of the second agent comprises a concentration range of between 0.1 μM and 110 μM.

8. The method of claim 1, wherein the at least one ALK is selected from the group consisting of ALK1, ALK2, ALK3, ALK4, ALK5, ALK6 and ALK7.

9. The method of claim 6, wherein the first agent and/or the second agent is selected from the group consisting of SB431542, DMH-1, and Alk5 inhibitor II.

10. The method of claim 1, wherein the at least one ALK is selected from the group consisting of ALK1, ALK2, ALK3, ALK4, ALK6 and ALK7.

11. The method of claim 1, wherein the at least one ALK is selected from the group consisting of SB431542 and DMH-1.

12. The method claim 6, wherein the first agent and/or the second agent is not ALK5 inhibitor II.

13. The method of claim 6, wherein the first agent and/or the second agent exhibits an IC₅₀ for the at least one ALK that is less than or equal to 500 nm or wherein the cells are contacted with a concentration of the first agent and/or the second agent that is equal to or greater than its IC50 value for at least one ALK.

14. The method claim 6, wherein the first agent and/or the second agent is more selective for the at least one ALK than for at least one mitogen activated protein kinase (MAPK) or other kinases.

15. The method of claim 1, wherein between at least 5% and 65% of the endocrine cells in the population differentiate into SC-β cells.

16. An isolated non-native SC-β cell or population thereof generated according to the method of claim 1 that exhibits a glucose stimulated insulin secretion (GSIS) response both in vitro and in vivo.

17. An isolated non-native SC-β cell or population thereof according to claim 16 that exhibits a stimulation index that is at least between 1.5-fold and 10-fold greater than the stimulation index of a control SC-β cell.

18. An isolated non-native SC-β cell or population thereof according to claim 16 that produces between approximately 300 uIU and 4000 uIU per 30 minute incubation at a high glucose concentration.

19. An isolated non-native SC-β cell or population thereof according to claim 16 that two weeks after transplantation into a subject in vivo releases between 3 uIU/mL and 81 uIU/mL of insulin within 30 minutes of administering 2 g/kg glucose to the subject.

20. A microcapsule comprising the isolated non-native SC-β cell or population thereof according to claim 16 encapsulated therein.

21. A microencapsulation device comprising the isolated non-native SC-β cell or population thereof according to claim 16 encapsulated therein.

22. A method for the treatment of a subject in need thereof, the method comprising administering to a subject in need thereof an isolated population of non-native SC-β cells produced according to the method of claim 1.

23. An artificial islet or pancreas comprising of non-native SC-β cells produced according to the method of claim 1.
